(19) United States
(12) Patent Application Publication
(10) Pub. No.: US 2010/0075917 A1
(43) Pub. Date: Mar. 25, 2010

(54) THIONUCLEOSIDES AND PHARMACEUTICAL APPLICATIONS

(75) Inventors: Jean-Luc Decout, Vaulnaveys Le Haut (FR); Beatrice Gerland, Valence (FR); Jerome Desire, Tencin (FR); Michel Lepoivre, Bures Sur Yvette (FR); Jan Balzarini, Heverlee (BE)

Correspondence Address: CANTOR COLBURN, LLP, 20 Church Street, 22nd Floor, Hartford, CT, 06103, US

(73) Assignee: UNIVERSITE JOSEPH FOURIER (GRENOBLE 1), GRENOBLE (FR)

(21) Appl. No.: 12/447,346

(22) PCT Filed: Oct. 29, 2007
(86) PCT No.: PCT/FR2007/001784

§ 371 Date: Jul. 27, 2009

(30) Foreign Application Priority Data
Oct. 27, 2006 (FR) 06/09460

(51) Int. Cl. A61K 31/7064; C07H 19/06; A61P 35/00; A61P 31/18

(52) U.S. Cl. 514/50; 536/28.2

(57) ABSTRACT

The invention relates to a pharmaceutical composition containing as an active ingredient at least one compound selected from the compounds of the formula (II), in which the S atom is bonded to the nucleoside carbon at T or to the nucleoside carbon at 3′, and from the compounds of the following formula (III): formulae in which B is a nucleotide natural or modified base, x is 0, 1 or 2, and R1 and R represent a carbonated group or a molecular hydrocarbon remnant that can be substituted and/or interrupted by one or more atoms and/or by one or more groups containing one or more atoms, said atoms being selected from N, O, P, S, Si, X where X is halogen. The invention also relates to pharmaceutically acceptable carrier.

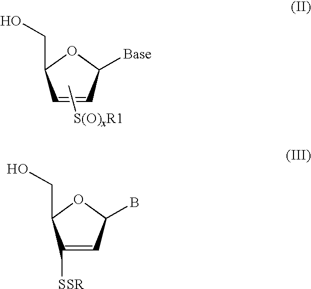

The present invention relates to thionucleosides and to their pharmaceutical uses.

One subject of the invention consists of a pharmaceutical composition comprising, as active principle, at least one compound chosen from:

the compounds corresponding to formula (II) below:

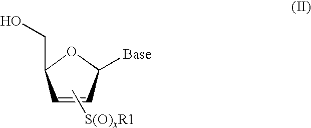

in which the S atom is bonded to the 2′ carbon or to the 3′ carbon of the nucleoside,

and the compounds corresponding to formula (III) below:

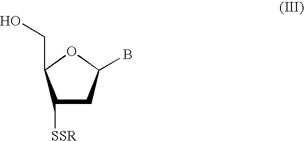

in which formulae

B represents a natural or modified purine or pyrimidine nucleotide base,

x is equal to 0, 1 or 2, and

R1 and R represent a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen,

said composition also comprising at least one pharmaceutically acceptable excipient.

The invention also relates to the use of the above compounds, and to those more specifically described hereinbelow as therapeutic active principle, or medicament. Such a medicament is in particular intended to incorporate a pharmaceutical composition according to the invention.

The invention lies in the use of such a composition in antiviral treatments, especially anti-HIV1 and anti-HIV2 treatments, and in anticancer treatments.

The compounds corresponding to formula (II) are advantageously compounds that satisfy the characteristics hereinbelow, these characteristics being considered individually or in combination with each other:

R1 is chosen from R3-Si(R4)(R5)(R6) in which R3 represents a hydrocarbon-based chain of two carbon atoms, which may be unsaturated and/or substituted, and R4, R5 and R6, which may be identical or different, each independently represent a hydrocarbon-based group; preferably, R3 represents therein CH₂—CH₂ and R4, R5 and R6 are identical and represent CH₃,

B is a nucleotide base chosen from natural or modified pyrimidine bases; a person skilled in the art has at his disposal in the literature modified bases, which he knows how to obtain and use, in particular such as analogs of natural bases (by way of example, reference may be made to the publication Frontiers in Nucleosides and Nucleic Acids, Editors R. F. Schinazi and D. C. Liotta, IHL Press, 2004, pp. 3-55); 5-bromouracil is an example thereof.

Preferential compounds according to the invention satisfy all the above characteristics, and in addition the S atom is bonded to the 2′ carbon of the nucleoside and x is equal to 0. Such compounds are especially chosen from the following compounds:
2′,3′-Didehydro-2′,3′-dideoxy-2′-(2-trimethylsilyl)ethylthiouridine 1
2′,3′-Didehydro-2′,3′-dideoxy-2′-(2-(trimethylsilyl)ethylthiothymidine 3
2′,3′-Didehydro-2′,3′-dideoxy-2′-(2-trimethylsilyl)ethylthiocytidine 5.

Other compounds of interest according to the invention satisfy all the above characteristics, and in addition the S atom is bonded to the 3′ carbon of the nucleoside and x is equal to 0. The compound 2′,3′-didehydro-2′,3′-dideoxy-3′-(2-trimethylsilyl)ethylthiothymidine 4 is a preferred example thereof.

Other preferential compounds of the invention satisfy all the above characteristics, and in addition the S atom is bonded to the 2′ carbon of the nucleoside and x is equal to 1. Such compounds are especially chosen from the following compounds:
2′,3′-didehydro-2′,3′-dideoxy-2′-(2-trimethylsilyl)ethylthiothymidine sulfoxide 6
2′,3′-didehydro-2′,3′-dideoxy-2′-(2-trimethylsilyl)ethylthiouridine sulfoxide 8.

Yet other advantageous compounds of the invention satisfy all the above characteristics, and in addition the S atom is bonded to the 2′ carbon of the nucleoside and x is equal to 2. A preferred compound is 2′,3′-didehydro-2′,3′-dideoxy-2′-(2-trimethylsilyl)ethylthiothymidine sulfone 7.

Other compounds of interest according to the invention are non-silyl compounds of formula (II) in which the S atom is bonded to the 2′ carbon of the nucleoside, x is equal to 0 and R1 represents an alkyl group. They are illustrated by the following compounds: 2′,3′-didehydro-2′,3′-dideoxy-2′-(3-methylbutyl)thiouridine 2 and 2′,3′-didehydro-2′,3′-dideoxy-2′-methylthiouridine 31.

The structure of the compounds identified by a figure or a number is represented at the end of the description and their properties are illustrated in the examples hereinbelow.

Other compounds corresponding to formula (II) are advantageously compounds that satisfy the characteristics hereinbelow, these characteristics being considered individually or in combination with each other:

x is equal to 0 and R1 represents SR2 in which R2 represents a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen; it is advantageously chosen from ortho-nitrophenyl, para-nitrophenyl and trichloromethyl groups,

B is a nucleotide base chosen from natural or modified pyrimidine bases, as defined above.

When the S atom is bonded to the 2′ carbon of the nucleoside, the preferred compounds are chosen from:
2′,3′-didehydro-2′,3′-dideoxythymidin-2′-yl trichloromethyl disulfide 12
2′,3′-didehydro-2′,3′-dideoxyuridin-2′-yl trichloromethyl disulfide 13
2′,3′-didehydro-2′,3′-dideoxythymidin-2′-yl 4-nitrophenyl disulfide 14
2′,3′-didehydro-2′,3′-dideoxythymidin-2′-yl 2-nitrophenyl disulfide 15
2′,3′-didehydro-2′,3′-dideoxyuridin-2′-yl 4-nitrophenyl disulfide 16
2′,3′-didehydro-2′,3′-dideoxyuridin-2′-yl 2-nitrophenyl disulfide 17.

The compounds corresponding to formula (II) above have an anti-proliferative and/or cytotoxic effect, and may be intended for treating cancer. Thus, according to the invention, they will advantageously be used for obtaining a medicament in an anticancer treatment.

The compounds of the invention corresponding to formula (III) are advantageously compounds that satisfy the characteristics hereinbelow, these characteristics being considered individually or in combination with each other:

B represents a natural or modified pyrimidine base, as defined above,

R is preferably chosen from CH₂—CH═CH₂, alkyl groups, for example CH₃, C₄H₉ or C₈H₁₇, hydroxyalkyl groups, for example CH₂CH₂OH or C₆HH₁₂OH, CH₂CH₂NH₂.HCl, and ortho-nitrophenyl, para-nitrophenyl and trichloromethyl groups.

Thus, such a compound may be chosen from the following compounds:
2′,3′-dideoxycytidin-3′-yl methyl disulfide 19
3′-deoxythymidin-3′-yl allyl disulfide 20
3′-deoxythymidin-3′-yl 2-hydroxyethyl disulfide 21
3′-deoxythymidin-3′-yl trichloromethyl disulfide 22
5-bromo-2′,3′-dideoxyuridin-3′-yl methyl disulfide 23
3′-deoxythymidin-3′-yl butyl disulfide 24
3′-deoxythymidin-3′-yl 4-nitrophenyl disulfide 25
3′-deoxythymidin-3′-yl 2-nitrophenyl disulfide 26
3′-deoxythymidin-3′-yl 2-aminoethyl disulfide hydrochloride 27
3′-deoxythymidin-3′-yl octyl disulfide 28
3′-deoxythymidin-3′-yl hexyl disulfide 29
3′-deoxythymidin-3′-yl 6-hydroxyhexyl disulfide 30.

The compounds corresponding to formula (III) above have an application in the treatment of a viral infection, in particular of an infection by HIV1 or HIV2. According to the invention, they will advantageously be used for obtaining a medicament in an antiviral treatment, such as an anti-HIV1 or anti-HIV2 treatment.

The invention also relates to novel nucleoside disulfide compounds corresponding to formula (I) below:

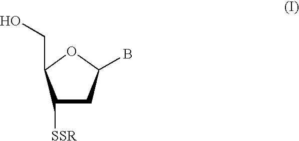

in which:

B represents thymine and R is chosen from CH₂—CH═CH₂, C₄H₉, CH₂CH₂OH and CH₂CH₂NH₂.HCl.

Disulfide compounds of formula (I) above in which B is a nucleotide base chosen from uracil, cytosine and 5-bromouracil and R represents CH₃ are also described.

Some of these compounds are described at the end of the description under the references 19, 20, 21, 23, 24 and 27.

The invention also relates to a process for preparing a compound corresponding to formula (IV) below:

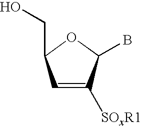

in which B, x and R1 are as defined previously for formula (II).

According to this process:

a compound corresponding to formula (II) above is provided, in which R1 represents R3-Si(R4)(R5)(R6) where R3 represents a hydrocarbon-based chain of two carbon atoms, which may be unsaturated and/or substituted, and R4, R5 and R6, which may be identical or different, each independently represent a hydrocarbon-based group;

said compound is reacted with a compound of formula RSX in which X represents a halogen and R represents a carbon-based group or a hydrocarbon-based molecular residue that may be substituted and/or interrupted with one or more atoms and/or with one or more groups comprising one or more atoms, said atoms being chosen from N, O, P, S, Si, X in which X represents a halogen, to obtain a disulfide;

the disulfide is reduced to a sulfide; and

the sulfide obtained is reacted with a compound R′X in which R′ represents a carbon-based group or a hydrocarbon-based molecular residue that may be substituted and/or interrupted with one or more atoms and/or with one or more groups comprising one or more atoms, said atoms being chosen from N, O, P, S, Si, X in which X represents a halogen; and X represents a halogen.

The synthetic scheme of this process according to the invention, which is illustrated in the experimental section of the description, is given hereinbelow, with R1 representing CH₂CH₂Si(CH₃)₃:

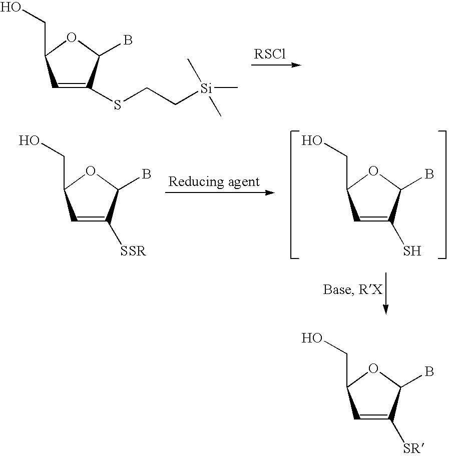

Another subject of the invention is a general synthetic process for obtaining disulfide compounds such as sulfur-containing amino acids, according to the synthetic scheme hereinbelow, which is illustrated in the experimental section of the description.

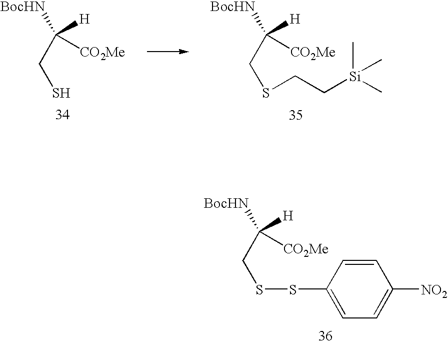

The invention is described hereinbelow in greater detail with the aid of the examples illustrating the synthetic processes that may be performed by a person skilled in the art to obtain the above compounds, and also the pharmaceutical properties thereof.

### I—PROCESS FOR OBTAINING THE COMPOUNDS OF THE INVENTION

The saturated silyl sulfide compounds serving as starting reagents in the following syntheses, and whose preparation is not described, may be obtained by means of processes known to those skilled in the art (C. Chambert et al., J. Org. Chem., 2000, 65, 249; Stamm, J. Org. Chem., 1963, 3264; Mahadevan et al., Synth. Commun., 1994, 3099; C. Chambert et al., J. Org. Chem., 2002, 67, 1898-1904).

### 2′,3′-Dideoxy-2′-(3-methylbutyl)thiouridine 2

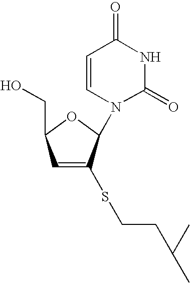

To a solution of 2′-deoxy-2′-(3-methylbutyl)thiouridine 33 (0.5 g; 1.51 mmol) in anhydrous pyridine (20 mL), maintained at 0° C. under argon, are added dimethoxytrityl chloride (1.023 g; 3 mmol) and 4-(dimethylamino)pyridine DMAP (18.3 mg; 0.15 mmol). The mixture is stirred for 15 minutes and then allowed to warm to room temperature, and stirred for 5 hours. The solvents are then evaporated off and co-evaporated with toluene. The residue obtained is taken up in a mixture of dichloromethane/methanol-2% triethylamine and chromatographed on silica gel with a 98/2 dichloromethane/methanol-2% triethylamine mixture.

The trityl product obtained is dissolved in anhydrous pyridine (7 mL) at 0° C. under argon for 15 minutes. Mesyl chloride (0.15 mL; 1.92 mmol) is then added and the mixture is stirred for 15 hours. The resulting mixture is neutralized by adding water (5 mL) at 0° C. and is stirred for 30 minutes. The solvents are evaporated off and co-evaporated with toluene.

The trityl-mesyl compound is treated with 2% dichloroacetic acid DCA in dichloromethane (25 mL) for 15 minutes. The reaction mixture is neutralized by adding 5% NaHCO₃ (300 mL) and the residue is washed with dichloromethane. The combined organic phases are dried over sodium sulfate and then concentrated to give the detritylated mesyl product (0.245 g; 0.6 mmol; 40%).

This product is then dissolved in acetonitrile (1.5 mL) under argon for 15 minutes, and K₂CO₃ (0.06 g; 0.43 mmol) is then added. The reaction mixture is stirred for 19 hours at 60° C. It is then filtered to remove the mineral salts, rinsing with methanol. The filtrate is evaporated to dryness and the product obtained is chromatographed on silica gel in a dichloromethane/methanol mixture (95/5). 2′,3′ Dideoxy-2′-(3-methylbutyl)thiouridine 2 is thus obtained in the form of white crystals (0.082 g; 0.26 mmol, 76.5%).

m.p.: 131-133° C.

¹H NMR (200 MHz, CDCl₃) δ 8.76 (1H, s, 3-H); 7.58 (1H, d, J=8.1 Hz, 6-H); 6.9 (1H, dd, J=1.5 Hz, J=3.2 Hz, 1′-H); 5.81 (1H, t, J=1.6 Hz, 3′-H); 5.68 (1H, d, J=8.1 Hz, 5-H); 4.95 (1H, m, 4′-H); 3.91 (1H, dd, J=2.6 Hz, J=12.6 Hz, 5′-H); 3.73 (1H, dd, J=3.2 Hz, J=12.52 Hz, 5′-H); 2.81 (2H, m, S—CH₂); 1.73 (1H, m, CH—(CH₃)₂); 1.55 (2H, m, (CH₂—CH)); 0.93 (6H, d, J=6.4 Hz, (CH₃—CH)).

¹³C NMR (50 MHz, CDCl₃) δ 163.27 (CO); 150.64 (CO); 141.0; 134.9; 123.68; 102.67; 90.33; 87.37; 63.54 (5′-CH₂); 37.23 (S—CH₂); 30.46 (CH₂—CH); 27.5; 22.18 ((CH₃)₂).

MS (FAB+, glycerol) m/z 313 [M+H]⁺

### 2′,3′-Didehydro-2′,3′-dideoxy-2′-(2-trimethylsilyl)ethylthiothymidine sulfoxide 6

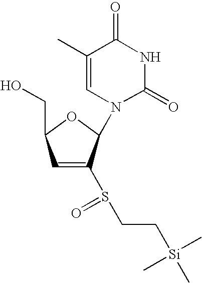

This compound is prepared from 2′-deoxy-2′-(2-(trimethylsilyl)-ethyl)thiothymidine 37, the synthesis of which is described hereinbelow:

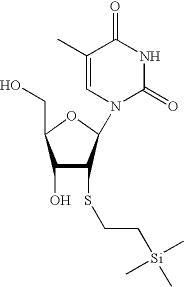

To a suspension of 2,2′-anhydrothymidine (5 g; 22 mmol) and anhydrous potassium carbonate (11 g; 79 mmol) in DMF (110 mL) is added 2-(trimethylsilyl)-ethanethiol (3.5 g; 26 mmol). The solution is stirred under argon at 120° C. for 3 hours. After filtering off and rinsing the mineral salts with dichloromethane, the solvents are evaporated off under reduced pressure to give a yellow oil. This residue is chromatographed on silica gel in a dichloromethane/methanol mixture (9515). The sulfide 37 is obtained in the form of a white solid (6.9 g; 19 mmol; 88%).

m.p.: 58-60° C.

¹H NMR (400 MHz, CDCl₃) δ 8.73 (1H, s, NH); 7.25 (1H, d, J=8.0 Hz, 6-H); 5.46 (1H, d, J=9.2 Hz, 1′-H); 4.36 (1H, m, 3′-H); 4.24 (1H, m, 4′-H); 3.99 (2H, m, 2′H+5′-H); 3.81 (1H, m, 5′-H); 2.60 (2H, m, S—CH₂); 1.96 (3H, s, 5-CH3); 0.85 (2H, m, CH₂Si); −0.02 (9H, s, Si(CH₃)₃).

¹³C NMR (100 MHz, CDCl₃) δ 163.4 (C2); 150.5 (C4); 138.87 (C6); 111.4 (C5); 93.3 (C1′); 86.6 (C4′); 71.5 (C3′); 63.1 (5′-CH₂); 52.8 (C2′); 28.4 (S—CH₂); 18.1 (CH₂—Si); 12.4 (CH₃); −1.8 (Si(CH₃)₃).

MS (DCI, NH₃-isobutane) m/z 375 [M+H]⁺, 392 [M+H+NH₃]⁺.

To a solution of 2′-deoxy-2′-(2-(trimethylsilyl)ethyl)thiothymidine 37 (0.05 g, 0.14 mmol) in methanol (5 mL) is added the tetrabutylammonium salt of oxone (100 mg, 0556 mmol) in 25-mg portions (0.5 equivalent) every 30 minutes (analyses by TLC). The mixture is stirred for 15 hours and is then evaporated to dryness and chromatographed on silica gel in a dichloromethane/methanol mixture (95/5). The unsaturated sulfoxide 6 is thus obtained in the form of a white powder (30 mg; 0.008 mmol, 58%).

¹H NMR (400 MHz, CDCl₃) δ 8.69 (1H, s, NH); 7.89 (1H, s, 6-H); 6.90 (1H, m, 1′-H); 6.89 (1H, m, 3′-H); 5.11 (1H, m, 4′-H); 3.99 (2H, m, 5′-H×2); 3.07-2.76 (2H, m, S—CH₂); 1.88 (3H, s, 5-CH₃); 1.10-0.94 (2H, m, CH₂Si); 0.02 (9H, s, Si(CH₃)₃).

¹³C NMR (100 MHz, CDCl₃) δ 163.7 (C2); 150.5 (C4); 140.1 (C2′); 137.8 (C3′); 135.8 (C6); 111.7 (C5); 87.6 (C1′+C4′); 62.9 (5′-CH₂); 48.1 (S—CH₂); 12.4 (CH₃); 6.7 (CH₂—Si); −1.9 (Si(CH₃)₃).

MS (DCI, NH₃-isobutane): m/z 373 [M+H]⁺.

### 2′,3′-Didehydro-2′,3′-dideoxy-2′-(2-trimethylsilyl)ethylthiothymidine sulfone 7

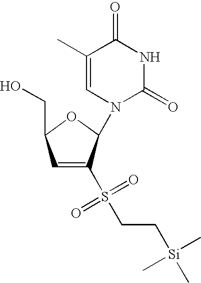

To a solution of 2′-deoxy-2′-(2-(trimethylsilyl)ethyl)thiothymidine 37 (0.02 g, 0.06 mmol) in anhydrous dichloromethane (1 mL) is added meta-chloroperbenzoic acid mCPBA (15 mg, 0.09 mmol). The mixture is stirred under argon for 15 hours and is then evaporated to dryness and chromatographed on silica gel with a dichloromethane/methanol mixture (95/5). The unsaturated sulfone 7 is thus obtained in the form of a white powder (15 mg; 0.004 mmol, 68%).

m.p.: 78-80° C.

¹H NMR (400 MHz, CDCl₃) δ 7.60 (1H, s, 6-H); 7.18 (1H, m, 1′-H); 6.18 (1H, m, 3′-H); 5.08 (1H, m, 4′-H); 3.97 (2H, m, 5′-H×2); 3.03 (2H, m, S—CH₂); 1.88 (3H, s, 5-CH₃); 0.98-0.87 (2H, m, CH₂Si); 0.02 (9H, s, Si(CH₃)₃).

¹³C NMR (100 MHz, CDCl₃) δ 162.5 (C2); 150.9 (C4); 145.5 (C2′); 138.8 (C3′); 135.8 (C6); 110.5 (C5); 91.4 (C1′); 85.8 (C4′); 62.8 (5′-CH₂); 50.9 (S—CH₂); 13.1 (CH₃); 8.5 (CH₂—Si); −2.1 (Si(CH₃)₃).

MS (DCI, NH₃-isobutane): m/z 389 [M+H]⁺.

### 2′,3′-Didehydro-2′,3′-dideoxy-2′-(2-trimethylsilyl)ethylthiouridine sulfoxide 8

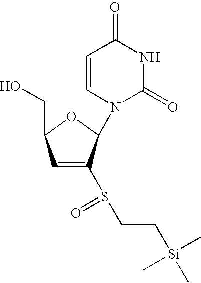

This compound is prepared from 2′-deoxy-2′-(2-(trimethylsilyl)ethyl)-thiouridine 37.

To a solution of 2′-deoxy-2′-(2-(trimethylsilyl)ethyl)thiouridine 37 (0.1 g, 0.29 mmol) in methanol (5 mL) is added the tetrabutylammonium salt of oxone (208 mg, 0.58 mmol) in 52-mg portions (0.5 equivalent) every 30 minutes (analyses by TLC). The mixture is stirred for 15 hours and is then evaporated to dryness and chromatographed on silica gel with a dichloromethane/methanol mixture (95/5). The unsaturated sulfoxide 8 is thus obtained in the form of a white powder (70 mg; 0.019 mmol, 64%).

¹H NMR (400 MHz, CDCl₃) δ 8.19 (1H, d, J=8.0 Hz, 6-H); 6.87 (1H, m, 3′-H); 6.83 (1H, m, 1′-H); 5.77 (1H, d, J=8.0 Hz, 5-H); 5.12 (1H, m, 4′-H); 3.97 (2H, m, 5′-H×2); 3.07-2.84 (2H, m, S—CH₂); 1.05-077 (2H, m, CH₂Si); 0.08 (9H, s, Si(CH₃)₃).

¹³C NMR (100 MHz, CDCl₃) δ 163.5 (C2); 150.5 (C4); 143.9 (C6); 140.4 (C2′); 103.9 (C3′); 103.0 (C5); 87.9 (C1′); 87.7 (C4′); 62.7 (5′-CH₂); 53.4 (S—CH₂); 6.6 (CH₂—Si); −1.9 (Si(CH₃)₃).

MS (DCI, NH₃-isobutane): m/z 352 [M+H]⁺.

### 2′,3′-Dideoxycytidin-3′-yl methyl disulfide 19

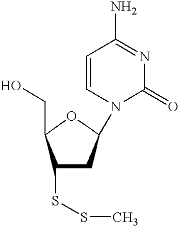

To a solution of 2′,3′-dideoxy-3′-(2-trimethylsilyl)ethylthiocytidine (0.05 g; 0.014 mmol) and methyl disulfide (320 μL; 3.55 mmol) in anhydrous THF (2 mL) is added dimethyl(thiomethyl)sulfonium tetrafluoroborate (36 mg; 0.023 mmol). The mixture is stirred for 48 hours under argon, and sodium bicarbonate solution (10%; 100 μL) is then added. After evaporating to dryness, the residue obtained is dissolved in a minimum amount of water and chromatographed on a C18 cartridge (1 g) with a water/methanol mixture (9/1) and, after evaporating to dryness, is then taken up in a minimum amount of eluent and chromatographed on silica gel with a dichloromethane/methanol mixture (90/10) to give the methyl disulfide 19 (37 mg; 0.128 mmol; 92%) in the form of a white foam.

¹H NMR (400 MHz, MeOD) δ 8.17 (1H, d, J=7.6 Hz, 6-H); 6.15 (1H, dd, J=6.8 Hz, J=4 Hz, 1′-H); 5.92 (1H, d, J=7.6 Hz, 5-H); 4.03 (1H, m, 4′-H); 3.95-3.82 (2H, m, 5′-H×2); 3.53 (1H, m, 3′-H); 2.67 (1H, m, 2′-H); 2.42 (1H, m, 2′-H); 2.48 (3H, s, CH₃—S),

¹³C NMR (100 MHz, MeOD) δ 163.4 (C4); 150.2 (C2); 143.7 (C6); 96.4 (C5); 88.0 (C1′); 87.9 (C4′); 62.5 (5′-CH₂); 46.8 (C3′); 40.9 (2′-CH₂); 25.3 (S—CH₃).

MS (FAB+, glycerol) m/z=290 [M+H]⁺; 312 [M+Na]⁺.

### 5-Bromo-2′,3′-dideoxyuridin-3′-yl methyl disulfide 23

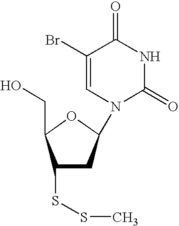

This compound is prepared from 2′,3′-dideoxy-3′-(2-(trimethylsilyl)-ethyl)thiouridine 38, the synthesis of which is described below.

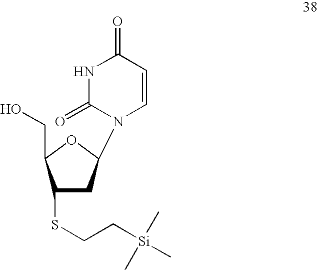

To a suspension of sodium hydride (60%; 234 mg; 7.02 mmol) in anhydrous DMF (8 mL) is added a solution of 2-(trimethylsilyl)ethanethiol (980 μL; 7.02 mmol) in DMF (8 mL). This mixture is stirred under argon for 15 minutes, and derivative 51 (3 g; 5.85 mmol) is then added. After 24 hours under argon at 90° C., the unreacted sodium hydride is neutralized with 3 mL of methanol and the solvents are evaporated off under reduced pressure. The residue is then taken up in dichloromethane (100 mL) and the solution is neutralized with NaH₂PO₄ solution (10%; 10 mL), 1 washed with water (100 mL) and dried over sodium sulfate, and then evaporated to dryness. The residue is taken up in dichloromethane and chromatographed on silica gel with a dichloromethane/ethyl acetate mixture (8/2) containing 1% triethylamine, to give the sulfide in the form of a yellow foam.

The pale yellow foam obtained is dissolved in a solution of dichloroacetic acid in dichloromethane (2%; 80 mL). The orange solution obtained is stirred under argon for 4 hours and then neutralized with sodium bicarbonate solution (5%; 30 mL). The aqueous phase is extracted with dichloromethane (50 mL) and the organic phases are combined and dried over sodium sulfate, and then evaporated to dryness. The residue is taken up in dichloromethane and chromatographed on silica gel with a dichloromethane/ethyl acetate mixture (6/4) to give the sulfide 38 (1.06 g; 3.08 mmol; 6% (2 steps)) in the form of a white solid.

m.p.: 148° C.

¹H NMR (400 MHz, CDCl₃) δ 9.47 (1H, s, NH); 7.82 (1H, d, J=8.4 Hz, 6-H); 6.12 (1H, dd, J=7.0 Hz, J=3.6 Hz, 1′-H); 5.73 (1H, d, J=8.4 Hz, 5-H); 4.05 (1H, m, 3′-H); 3.85 (2H, m, 5′-H×2); 3.47 (1H, m, 4′-H); 2.64-2.50 (4H, m, 2′-H×2, S—CH₂); 0.84 (2H, m, CH₂—Si); −0.02 (9H, s, Si(CH₃)₃).

¹³C NMR (100 MHz, CDCl₃) δ 163.9 (CO); 150.4 (CO); 140.9; 101.9; 86.2; 85.7; 61.0 (5′-CH₂); 40.7; 40.1 (2′-CH₂); 27.5 (S—CH₂); 17.4 (CH₂—Si); −1.8 (Si(CH₃)₃).

MS (FAB+, glycerol) m/z=345 [M+H]⁺.

Microanalysis for C₁₄H₂₄N₂O₄Ssi.0.33H₂O:

Calculated C, 47.97; H, 7.09; N, 7.99; S 9.15.

Found C, 47.82; H, 7.13; N, 7.79; S 9.66.

To a solution of the silyl nucleoside 38 (0.150 g; 0.44 mmol) in anhydrous dichloromethane (4 mL) is added cyanogen bromide (0.230 mg; 2.17 mmol). The mixture is stirred under argon for 96 hours at 40° C. The symmetrical disulfide gradually appears in the form of a beige-colored precipitate, while the second product remains in solution. After hydrolysis with a phosphate buffer solution (0.5 M; pH 7; 2 mL) for 30 minutes, the solvents are evaporated to dryness. The residue obtained is taken up in a minimum amount of dichloromethane and chromatographed on silica gel with a dichloromethane/methanol mixture (98/2 and then 95/5). A symmetrical disulfide is obtained in the form of a white powder (27 mg; 0.04 mmol; 21%), and the desired bromosilyl derivative 39 below is obtained in the form of a white powder (42 mg, 0.1 mmol, 25%).

Bromosilyl Derivative 39

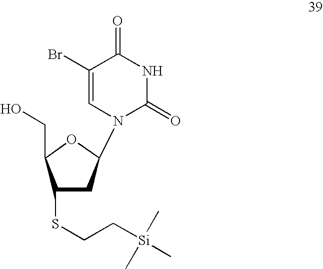

¹H NMR (400 MHz, CDCl₃) δ 8.41 (1H, s, 6-H); 6.09 (1H, dd, J=6.7 Hz, J=3.2 Hz, 1′-H); 4.14 (1H, s, 5′-H); 4.93 (2H, m, 4′-H and 5′-H); 3.95 (1H, m, 3′-H); 2.66 (2H, m, S—CH₂); 2.60-2.44 (2H, m, 2′-H×2); 1.28 (1H, t, 5′-OH); 0.88 (2H, m, CH₂—Si); −0.02 (9H, s, Si(CH₃)₃).

¹³C NMR (100 MHz, CDCl₃) δ 158.8 (C2); 149.3 (C4); 140.4 (C6); 96.1 (C5); 86.3 (C1′); 86.2 (C4′); 60.8 (5′-CH₂); 41.2 (2′-CH₂); 39.6 (C3′); 27.6 (S—CH₂); 17.5 (CH₂—Si); −1.5 (Si(CH₃)₃).

MS (FAB+, NBA) m/z=423 [M+H]⁺.

To a solution of 5-bromo-2′,3′-dideoxy-3′-(2-trimethylsilyl)ethylthiouridine 39 (0.02 g; 0.005 mmol) and methyl disulfide (170 μL; 1.90 mmol) in anhydrous THF (500 μL) is added dimethyl(thiomethyl)sulfonium tetrafluoroborate (22 mg; 0.014 mmol). The mixture is stirred for 24 hours under argon, and sodium bicarbonate solution (10%; 100 μL) is then added. After evaporating to dryness, the residue obtained is dissolved in a minimum amount of water and chromatographed on a C18 cartridge (1 g) with a water/methanol mixture (9/1) and, after evaporating to dryness, is then taken up in a minimum amount of eluent and chromatographed on silica gel with a dichloromethane/methanol mixture (95/5) to give the methyl disulfide 23 (11 mg, 0.003 mmol, 65%) in the form of a white foam.

¹H NMR (400 MHz, MeOD) δ 8.67 (1H, s, 6-H); 6.11 (1H, dd, J=6.8 Hz, J=3.5 Hz, l'-H); 4.01 (2H, m, 4′-H and 5′-H); 3.84 (1H, m, 5′-H); 3.62 (1H, m, 3′-H); 2.64-2.52 (2H, m, 2′-H×2); 2.48 (3H, s, S—CH₃).

¹³C NMR (100 MHz, MeOD) δ 160.3 (C2); 150.1 (C4); 140.7 (C6); 95.2 (C5); 86.0 (C4′); 85.3 (C1′); 59.7 (5′-CH₂); 44.1 (C3′); 39.5 (2′-CH₂); 23.2 (S—CH₃).

MS (DCI, NH₃-isobutane): m/z 369 [M+H]⁺.

### 2′-Deoxy-2′-(3-methylbutyl)thiouridine 33

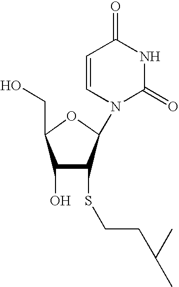

To a suspension of 2,2′-anhydrouridine (0.5 g; 2.2 mmol) and anhydrous potassium carbonate (1.1 g; 7.9 mmol) in dimethylformamide DMF (11 mL) is added 3-methylbutanethiol (0.271 g; 2.6 mmol). The solution is stirred under argon at 120° C. for 5 hours. After filtering off and rinsing the mineral salts with dichloromethane, the solvents are evaporated off under reduced pressure to give a yellow oil. This residue is taken up in dichloromethane/methanol (98/2) and chromatographed on silica gel in a dichloromethane/methanol mixture (95/5). The sulfide 33 is obtained in the form of a white solid (0.387 g; 1.17 mmol; 54%).

m.p.: 66-67° C.

¹H NMR (200 MHz, DMSO-d₆) δ 11.33 (1H, s, 3-H); 7.86 (1H, d, J=8.1 Hz, 6-H); 6.0 (1H, d, J=8.8 Hz, 1′-H); 5.69 (1H, d, J=8.1 Hz, 5-H); 5.56 (1H, d, J=5.3 Hz, 3′-OH); 5.06 (1H, t, J=5.1 Hz, 5′-OH); 4.17 (1H, m, 3′-H); 3.86 (1H, m, 4′H); 3.55 (2H, m, 5′-H); 3.4 (1H, dd, J=5.2 Hz, J=8.75 Hz, 2′-H); 2.4 (2H, m, S—CH₂); 1.55 (1H, m, CH—(CH₃)₂); 1.3 (2H, m, (CH₂—CH)); 0.7 (6H, m, (CH₃—CH)).

¹³C NMR (50 MHz, DMSO-d₆) δ 162.74 (CO); 150.6 (CO); 140.3; 102.3; 87.67; 86.5; 72.08; 61.35 (5′-CH₂); 51.7; 38.5 (S—CH₂); 28.4 (CH₂—CH); 26.7; 21.9 ((CH₃)₂).

MS (FAB+, glycerol) m/z 331 [M+H]⁺, 219 [M-uracil]⁺

### II—PROPERTIES OF THE COMPOUNDS OF THE INVENTION

The biological properties of the compounds of the invention and the pharmaceutical applications resulting therefrom are outlined below.

These properties were demonstrated in tests in which they showed an anti-proliferative effect and a cytotoxic effect for use in an anticancer treatment, and also in tests in which they showed an antiviral effect.

### Properties in an Anticancer Application

### 1. Description of the Tests

### 1.1 Antiproliferative Effects

The test compounds are subjected to two tests, a first test for measuring their influence on ribonucleotide reductase, and a second test for measuring their influence on the incorporation of tritiated thymidine.

11.1. Measurement of the Intracellular Concentrations of 2′-deoxyribonucleotides (dNTP)

The protocol adopted for performing this test (Roy, B.; Beuneu, C.; Roux, P.; Buc, H.; Lemaire, G.; Lepoivre, M. Simultaneous determination of pyrimidine or purine deoxyribonucleoside triphosphates using a polymerase assay. Anal. Biochem., 1999, 269, 403-409) consists in culturing human CEM-SS T lymphoma cells in the presence of the test compounds. The concentrations of dNTP and in particular of dATP are measured after 24 hours of incubation. dATP is the nucleoside triphosphate whose concentration decreases the most during tests with standard ribonucleotide reductase inhibitors such as hydroxyurea, which was used as control.

The quantification is related to the number of live cells counted after staining with trypan blue or related to the initial number of cells (approximately two million). It is measured as a percentage relative to a control manipulation.

### 1.1.2. Tests of Incorporation of Tritiated Thymidine

These tests are performed on the L1210 cell line (mouse lymphoma) according to a published protocol (Lepoivre M., Flaman J.-M., Bobé P., Lemaire G., Henry Y. J. Biol. Chem., 1994, 269, 21891-21897). The wells are inoculated (10 000 cells per well) and, after culturing for 72 hours, [³H]thymidine and the test compound are added to the culture medium. After incubation for 8 hours, the DNA is separated from the other cell constituents and the amount of labeled thymidine incorporated is quantified and compared with a control (without compound).

### 1.2. Cytotoxic Effects

The cytotoxic effect of a compound is studied on human CEM cells and MCF-7 mammary carcinoma cells that are or are not resistant to gemcitabine, which is an anticancer nucleoside used in chemotherapy.

The cytotoxicity (CC₅₀) was estimated by measuring the reduction of 3-(4,5-dimethylthiazol-2-yl)-2,5-diphenyltetrazolium bromide (MTT), which is converted into insoluble violet formazan. To do this, 100 000 cells (human CEM-SS or MCF-7 lines) are inoculated per well in 100 μl of culture medium, in 96-well microtitration plates. After 24 hours, the test products are added in 100 μl and incubation is continued for 48 to 72 hours. MIT is then added in a proportion of 20 μl of a solution at 5 mg/ml in PBS. Three hours later, 100 μl of a DMF/acetic acid/HCl/SDS (sodium dodecyl sulfate) solution are added. After 3 hours at 37° C., the absorbance of the formazan is measured at 590 nm and compared with that of the untreated control.

### 2. Test Results

### 2.1. Antiproliferative Effects

2.1.1 Measurement of the Intracellular Concentrations of 2′-deoxyribonucleotides (dNTP)

The nucleoside 1 was tested under the following conditions.

The effects of compound 1 at 50 μM on the concentrations of 2′-deoxynucleoside 5′-triphosphates (dNTP) necessary for DNA synthesis are calculated from the dNTP concentrations measured after 24 hours and determined in pmol of dNTP/million cells. The percentages are expressed as a percentage of the control with an average of four controls, taking the counts into account, and are compared with that of an anticancer agent, hydroxyurea (HU), tested at a concentration of 63 μM.

As indicated in Table 1 below, compound 1 proved to be antiproliferative on CEM/SS T lymphoma cells by greatly decreasing the concentrations of nucleoside triphosphates required for DNA synthesis.

TABLE 1

| | | | | | Hydroxyurea control, |
|---|---|---|---|---|---|
| | | | | | 63 μM |
| dNTP | dATP | dGTP | dCTP | dTTP | dTTP |
| % (1 at 50 μM) | 50 | 31 | 42 | 85 | 51 |
| 24 hours | | | | | |

2.1.2. Decrease of the Incorporation of Tritiated Thymidine into L1210 Cells as a Percentage of the Control

The effects of compound 1 at 50 μM and 200 μM, respectively, on the concentrations of tritiated thymidine incorporated are expressed as a percentage of the control.

The results are given in Table 2.

TABLE 2

| | |
|---|---|
| 1 at 50 μM | 62 |
| 1 at 200 μM | 14 |

An inhibition (decrease of 38% and 86% for the 50 μM and 200 μM concentrations, respectively) of the incorporation of tritiated thymidine into the DNA is observed in the presence of derivative 1, which means that it inhibits this synthesis and thus acts on the cell proliferation.

### 2.2 Cytotoxic Effects

2.2.1 Compounds of Formula (II) in which R1 Represents CH₂CH₂Si(CH₃)₃

Compounds 1 and 3 to 8 corresponding to formula (II) above; compound 2 corresponding to the non-silyl compound, it corresponds to formula (II) above in which R1 represents CH₂CH₂CH(CH₃)₂ and was also tested to observe a possible effect of the silicon. These compounds were evaluated for their cytotoxicity on human CEM cells and MCF-7 mammary carcinoma cells resistant (MCM-7*) or non-resistant (MCF-7) to gemcitabine, which is an anticancer nucleoside used in chemotherapy.

The results are given in Table 3 below:

TABLE 3

| | Compounds | | | | | | | |
|---|---|---|---|---|---|---|---|---|
| | 3 | 1 | 2 | 5 | 4 | 7 | 6 | 8 |
| CC₅₀ μM | 13 | 156 | >>200 | 217 | 82 | 63 | >>200 | >>200 |
| CEM | | | | | | | | |
| CC₅₀ μM | 40 | 69 | >>200 | 41% | 33% | 120 | | |
| MCF-7 | | | | cytotoxicity | cytotoxicity | | | |
| | | | | at 200 μM | at 200 μM | | | |
| EC₅₀ μM | 37 | | | | | | | |
| MCF-7* | | | | | | | | |
| EC₅₀ μM | 41 | | | | | | | |
| MCF-7* | | | | | | | | |

2.2.2 Disulfide Compounds of Formula (II) in which x is Zero and R1 Represents SR2

The unsaturated disulfide compounds 11, 12 and 13 corresponding to the above formula showed cytotoxicity on human CEM cell lines and MCF-7 mammary carcinoma cell lines, as shown by the results in Table 4 below:

TABLE 4

| | Compound | | |
|---|---|---|---|
| | 11 | 12 | 13 |
| EC₅₀ μM | >>200 | 46 | 41 |
| CEM | | then plateau | then plateau |
| EC₅₀ μM | | | 85 |
| CEM | | | then plateau |
| EC₅₀ μM | | | 194 |
| MCF-7 | | | |
| EC₅₀ μM | | | 177 |
| MCF-7* | | | |
| EC₅₀ μM | | | 194 |
| MCF-7* | | | |

The unsaturated disulfide compounds 14, 15, 16 and 17 corresponding to formula (II) above showed a cytotoxic effect on human CEM T4 lymphocytes and human Molt4/C8 cells and murine leukemia cells (L1210) and murine mammary cancer cells (FM3A), as shown by the results in Table 5 below:

Human T4-lymphocyte CEM and Molt4/C8 cells, murine leukemia (L1210) and murine mammary carcinoma cells (FM3A)

TABLE 5

| Compound | L1210/0 | FM3A/0 | Molt4/C8 | CEM | HeLa |
|---|---|---|---|---|---|
| 14 | 31 ± 3 | 39 ± 3 | 12 ± 5 | 31 ± 1 | 7.4 ± 1.6 |
| 15 | 42 ± 3 | 175 ± 30 | 30 ± 15 | 28 ± 0 | 31 ± 1 |
| 16 | 42 ± 4 | 45 ± 2 | 28 ± 10 | 41 ± 1 | 16 ± 2 |
| 17 | 43 ± 5 | 206 ± 25 | 42 ± 1 | 40 ± 4 | 29 ± 9 |

### 2.2.3 Saturated Disulfide Compounds of Formula (I)

The disulfide compounds 18, 19, 20, 21, 22 and 23 corresponding to formula (I) above showed a cytotoxic effect on the CEM cells, as shown by the results in Table 6 below:

TABLE 6

Compound

| | 18 | 19 | 20 | 21 | 22 | 23 |
|---|---|---|---|---|---|---|
| EC₅₀ μM | 152 | >>200 | 48 | >>200 | 21 | 66 |
| CEM | | | | | | |
| EC₅₀ μM | | | 46 | | | |
| CEM | | | | | | |

The abovementioned disulfide compounds 18, 20, 21 and 22 and the disulfide compounds 24, 25, 26 and 27 showed a cytotoxic effect on L1210/0, FM3A/0, Molt4/C8, CEM and HeLa cells, as shown by the results in Table 7 below:

TABLE 7

| Compound | L1210/0 | FM3A/0 | Molt4/C8 | CEM | HeLa |
|---|---|---|---|---|---|
| 18 | 44 ± 1 | ≧500 | 66 ± 9 | 187 ± 1 | 56 ± 8 |
| 20 | 43 ± 5 | 299 ± 45 | 34 ± 6 | 128 ± 18 | 40 ± 7 |
| 24 | 32 ± 0 | 286 ± 24 | 28 ± 7 | 44 ± 1 | 23 ± 2 |
| 21 | 26 ± 3 | >500 | 128 ± 33 | 347 ± 71 | ≧100 |
| 25 | 32 ± 3 | 148 ± 16 | 29 ± 2 | 34 ± 0 | 28 ± 12 |
| 26 | 33 ± 7 | 220 ± 19 | 31 ± 5 | 32 ± 1 | 15 ± 0 |
| 22 | 45 ± 1 | 244 ± 49 | 55 ± 19 | 121 ± 18 | 39 ± 10 |
| 27 | 65 ± 9 | >500 | 163 ± 41 | 437 ± 71 | >100 |

It is observed that the cytotoxic effects are dependent on the nature of the side chain of the disulfides and on the cell lines.

### Antiviral Properties

### 1. Description of the Tests

The evaluation test is described in Roy, B.; Chambert, S.; Lepoivre M.; Aubertin, A.-M.; Balzarini, J.; Décout, J.-L., Deoxyribonucleoside 2′- or 3′-mixed disulfides: prodrugs to target ribonucleotide reductase and/or to inhibit HIV reverse transcription. J. Med. Chem. 2003, 46, 2565-2568.

The toxicity and the antiviral activities were measured at different concentrations, and their evolution as a function of the concentration makes it possible to evaluate:
the concentration at which 50% of the cells are dead in the absence of virus (CC 50) for each of the test compounds, from the change in absorbance at 540 nm of formazan as a function of the concentration of test compound,
the concentration of compound that leads to a 50% reduction in the activity of viral reverse transcriptase (EC 50). This concentration is determined from the curve representing the change in the percentage of “remaining reverse transcriptase” activity (defined as being the ratio of the activity of the reverse transcriptase in the presence of the test compound to that of the untreated cells) as a function of the concentration of test compound.

The compounds derived from the following families showed anti-HIV effects (HIV 1 and 2) with toxicity modulated by the substituent R.

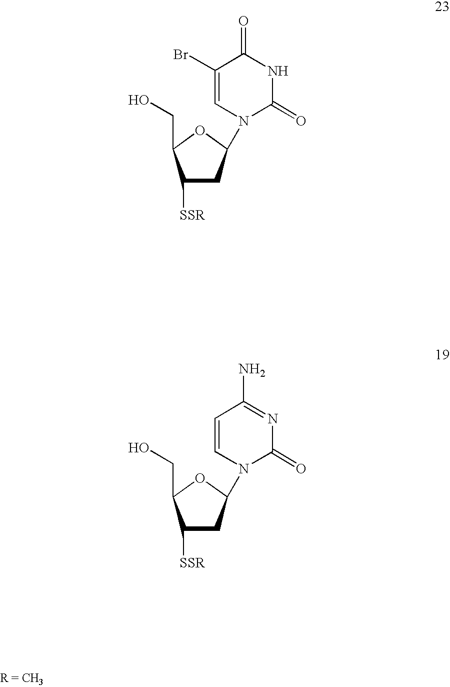

### 2. Test Results

### 2.1. Antiviral Effects

The compounds identified in the tables below were tested and showed anti-HIV-1 and anti-HIV-2 effects.

In the tables below:

EC₅₀: concentration required to afford 50% protection to the CEM cells against the cytopathogenicity of HIV;

CC₅₀: cytotoxic concentration or concentration required to reduce the viability of the CEM cells by 50%.

### First and Second Series

Compound

| | 18 | 19 |
|---|---|---|
| EC₅₀ μM | 4.0 ± 0.0 | 3.3 ± 2.3 |
| HIV-1 | Tenofovir: 5.5 ± 2.1 | Tenofovir 5.5 ± 2.1 |
| EC₅₀ μM | 6.5 ± 0.7 | 10.5 ± 9.3 |
| HIV-2 | Tenofovir: 2.6 ± 2.0 | Tenofovir: 2.6 ± 2.0 |

| | Compound | | | | | |
|---|---|---|---|---|---|---|
| | 18 | 20 | 24 | 21 | 22 | 27 |
| EC₅₀ μM HIV-1 | 10.3 ± 5.7 | 12.5 ± 3.5 | 8 ± 3 | 13.5 ± 3.0 | 25 ± 0 | 27.5 ± 3.5 |
| EC₅₀ μM | 11.7 ± 2.9 | 15 ± 7 | >10 | 14 ± 7 | 17.5 ± 3.5 | 27.5 ± 3.5 |
| HIV-2 | | | | | | |
| CC₅₀ μM | 107 ± 7 | 53 ± 7 | 23.3 ± 0.6 | >250 | 103 ± 4 | >250 |

### Third Series

| | Compound | | | | | |
|---|---|---|---|---|---|---|
| | 18 | 20 | 24 | 21 | 22 | 27 |
| EC₅₀ μM | 22.5 ± 3.5 | 17.5 ± 3.5 | >10 | 17.5 ± 3.5 | 20 ± 0 | 20 ± 0 |
| HIV-1 | | | | | | |
| EC₅₀ μM | 17.5 ± 11 | 20 ± 7 | >10 | 15 ± 7 | 27.5 ± 3.5 | 27.5 ± 3.5 |
| HIV-2 | | | | | | |
| *CEM/TK⁻ EC50 μM | >50 | >50 | >10 | >50 | 22.5 ± 3.5 | 150 ± 0 |
| HIV-2 | | | | | | |

Effect in CEM not expressing thymidine kinase activity (CEM/TK⁻, cells having no thymidine kinase activity).

| | Compound | | | | | |
|---|---|---|---|---|---|---|
| | 16 | 17 | 30 | 31 | 32 | 19 |
| EC₅₀ μM | 37.5 ± 17.7 | 6.5 ± 4.9 | 3.0 ± 0.0 | ≧10 | 15.0 ± 0.0 | 20 ± 7 |
| HIV-1 | | | | | | |
| EC₅₀ μM | 9.5 ± 7.8 | ≧10 | 4.5 ± 2.1 | 6.5 ± 0.7 | 15.0 ± 0.0 | 20 ± 7 |
| HIV-2 | | | | | | |
| *CEM/TK⁻ | 32.5 ± 25 | >10 | >10 | >10 | >50 | 45 ± 21 |
| EC₅₀ μM | | | | | | |
| HIV-2 | | | | | | |

The results mentioned in these tables show an anti-HIV-1 and anti-HIV-2 effect for the majority of the nucleosides tested. The strong decrease of the antiviral effects in the CEM/TK⁻ cells confirms that the nucleosides must be phosphorylated in vivo to be active. The toxicity of the active compounds depends greatly on the nature of the side chain borne by the disulfide function.

2.2. Inhibitory Effect on the Transformation of Mouse Embryo C3H/3T3 Cells Induced by Moloney Sarcoma Virus (Model of Infection with HIV)

| | Compound | | | | | | | | | | |
|---|---|---|---|---|---|---|---|---|---|---|---|
| | | | | | | | | | | (R)- | |
| | 18 | 19 | 20 | 24 | 21 | 25 | 26 | 14 | 15 | PMEA | PMEA |
| EC₅₀ | 8.3 ± 2.3 | 48 ± 2 | 2.2 ± 0.3 | 1.0 ± 0.5 | 5.9 ± 0.5 | 0.89 ± 0.36 | 1.1 ± 0.8 | 15 ± 0 | 8.3 ± 0.4 | 0.23 ± 0.03 | 0.53 ± 0.13 |
| μM | | | | | | | | | | | |
| MIC (μg/μM) | >100 | >100 | 100 | 100 | >100 | 20 (>4) | 20 | 100 | 100 | >10 | >20 |
| | | | (>20) | (>20) | | | (>4) | (>20) | (>20) | | |

EC₅₀: effective concentration 50%
MIC: minimum inhibitory concentration

These data confirm the pronounced anti-retroviral effect of the evaluated compounds.

### III—PROCESS FOR OBTAINING A COMPOUND OF FORMULA (IV) ACCORDING TO THE PROCESS OF THE INVENTION

The process described previously is illustrated below for the synthesis of 2′,3′-didehydro-2′,3′-dideoxy-2′-methylthiouridine 31.

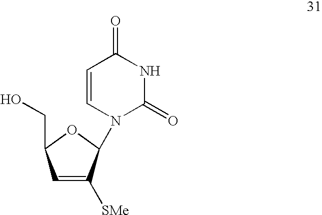

This compound was prepared from 2′,3′-didehydro-2′,3′-dideoxyuridin-2′-yl 4-nitrophenyl disulfide 40, whose synthesis from a stable sulfenyl halide is described below.

### 2′,3′-Didehydro-2′,3′-dideoxyuridin-2′-yl 4-nitrophenyl disulfide 40

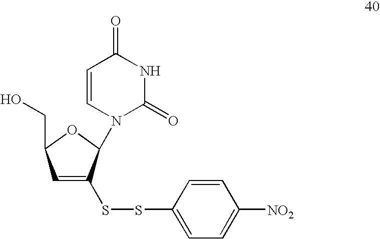

To a solution of 2′,3′-didehydro-2′,3′-dideoxy-2′-(2-trimethylsilyl)ethyl)-thiouridine (200 mg; 0.58 mmol) in anhydrous dichloromethane (6 mL), maintained under argon, is added 4-nitrobenzenesulfenyl chloride (333 mg; 1.75 mmol). The mixture is stirred for 15 hours and the solvent is then evaporated off. The residue obtained is taken up in a minimum amount of dichloromethane and chromatographed on silica gel in a dichloromethane/methanol mixture (98/2) and then (95/5) to give compound 40 (152 mg; 0.38 mmol; 66%) in the form of a yellow powder.

¹H NMR (400 MHz, CDCl₃) δ 9.40 (1H, s, NH); 8.16 (2H, m, Ar); 7.65 (1H, d, J=8.0 Hz, 6-H); 7.60 (2H, m, Ar); 7.04 (1H, m, 1′-H); 6.48 (1H, m, 3′-H); 5.70 (1H, d, J=8.4 Hz, 5-H); 4.99 (1H, 1s, 4′-H); 3.94 (1H, m, 5′-H); 3.83 (1H, m, 5′-H),

¹³C NMR (100 MHz, CDCl₃) δ 162.9 (C2); 150.3 (C4); 146.8 (C—NO₂); 143.9 (C—SS); 140.8 (C6); 135.1 (C2′); 131.8 (C3′); 126.6 (2 C, Ar); 126.3 (2 C, Ar); 102.8 (C5); 89.7 (C1′); 87.0 (C4′); 63.0 (5′-CH₂).

MS (DCI, NH₃-isobutane): m/z 396 [M+H]⁺; 413 [M+NH₃]⁺.

To a solution of 2′,3′-didehydro-2′,3′-dideoxyuridin-2′-yl 4-nitrophenyl disulfide 40 (30 mg, 0.08 mmol) in anhydrous methanol is added DTT (6.3 mg, 0.09 mmol). The solution is stirred for 30 minutes, and methyl iodide (50 μL, 0.8 mmol) is then added in the presence of NaHCO₃ (8 mg, 0.10 mmol). After 15 hours, the reaction mixture is evaporated and the residue is then chromatographed on silica gel in a dichloromethane/methanol mixture (95/5). The unsaturated methyl compound 31 is obtained in the form of a white solid (6 mg, 0.023 mmol, 31%).

¹H NMR (400 MHz, CDCl₃) δ 7.60 (1H, d, J=8 Hz, 6-H); 6.93 (1H, m, 1′-H); 5.76 (1H, s, 3′-H); 5.73 (1H, d, J=7.6 Hz, 5-H); 4.99 (1H, m, 4′-H); 3.94-3.76 (2H, m, J=3.2 Hz, J=11.6 Hz, 5′-H×2); 2.41 (Me).

¹³C NMR (100 MHz, CDCl₃) δ 162.8 (C2); 150.5 (C4); 141.8 (C6); 136.4 (C3′); 122.3 (C2′); 102.8 (C5); 90.2 (C1′); 87.2 (C4′); 63.6 (5′-CH₂); 15.1 (Me).

MS (DCI, NH₃-isobutane): m/z 257 [M+H]⁺.

### IV—OTHER PROCESS OF THE INVENTION

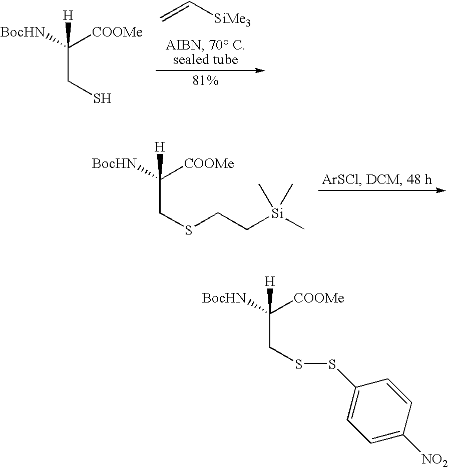

Silyl Sulfide in Cysteine Series 35

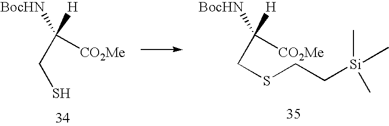

A solution of protected cysteine derivative 34 (1 g, 4.25 mmol) and of trimethylvinylsilane (740 mL, 5.1 mmol) is stirred in the presence of a catalytic amount (10%) of AIBN at 70° C. in a sealed tube for 24 hours. The reaction mixture is then evaporated to dryness to give compound 35 (1.1 g, 81%) in the form of a pale yellow oil.

¹H NMR CDCl₃ δ 5.40 (1H, d, NH), 4.52 (1H, m, Hα), 3.72 (3H, s, CH₃), 2.95 (2H, d, CH₂S), 2.52 (2H, m, CH₂S), 1.41 (9H, s, (CH₃)₃), 0.81 (2H, m, CH₂Si), 0.12 (9H, s, (CH₃)₃Si).

¹³C NMR CDCl₃ δ 171.5 (C═O), 155.0 (C═O), 79.8 (C Boc), 53.2, 52.3 (2 CH₂S), 34.2 (CH), 28.2 (3 CH₃ Boc), 17.2 (CH₂Si), −1.9 (3 CH₃Si).

HRMS for C₁₄H₂₉NO₄NaSSi [M+Na]⁺: theoretical: 358.1484; found: 358.1483

### Cysteine 4-nitrophenyl disulfide 36

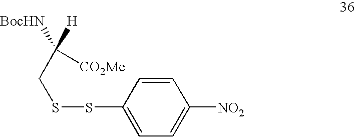

To a solution of silyl cysteine sulfide 35 (100 mg, 0.31 mmol) in anhydrous dichloromethane (5 mL) is added 4-nitrobenzenesulfenyl chloride (177 mg, 0.93 mmol) and the reaction medium is then stirred at room temperature under an inert atmosphere for 48 hours. The mixture is then diluted with dichloromethane, washed with water and then evaporated to dryness. The residue is purified by chromatography on silica gel in a cyclohexane/dichloromethane mixture (50/50 and then 70/30 and then 0/100) to give compound 36 (79 mg, 69%) in the form of a pale yellow oil.

¹H NMR CDCl₃ δ 8.22 (2H, d, arom. H), 7.66 (2H, d, arom. H), 5.31 (1H, m, NH), 4.62 (1H, m, Hα), 3.78 (3H, s, CO₂CH₃), 3.32 (1H, dd, CH₂), 3.19 (1H, dd, CH₂), 1.46 (9H, s, (CH₃)₃).

¹³C NMR CDCl₃ δ 107.7 (C═O), 154.8 (C═O), 146.4, 145.9 (2C arom.), 126.2, 124.1 (2×2C arom.), 80.5 (C(CH₃)₃), 52.7 (CO₂CH₃), 41.2 (CH₂S), 28.2 (3 CH₃).

The compounds identified in the description and the claims by their reference are described hereinbelow.

### Compounds Of Formula (II)

### Unsaturated Sulfide Compounds

The silyl compounds 1 and 3-8 are silylated, compounds 2 and 31 are not silylated.

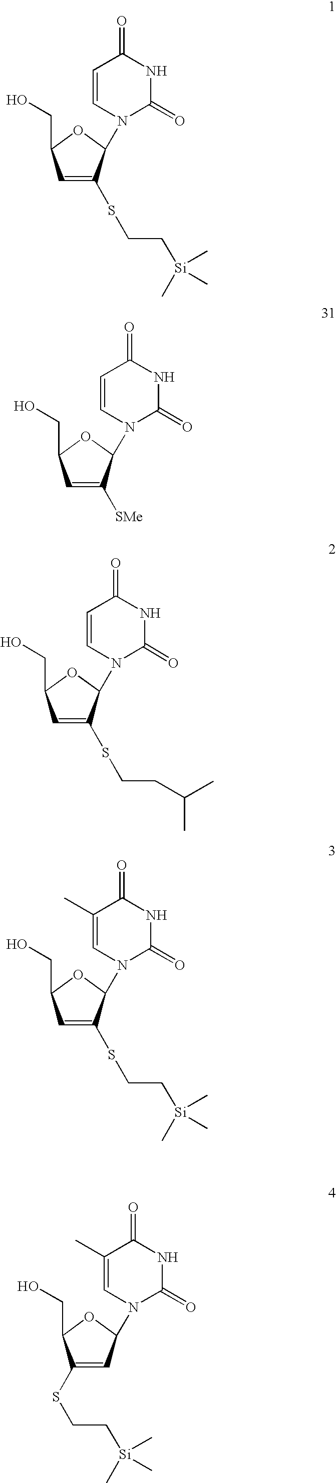

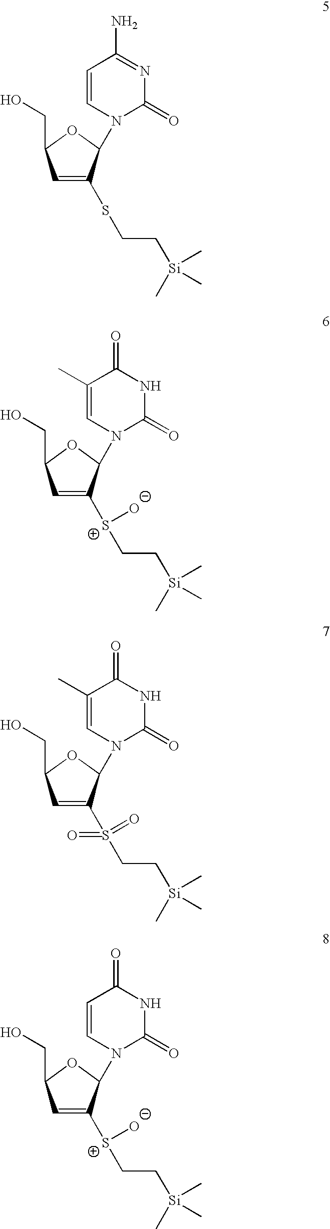

### Unsaturated Disulfide Compounds

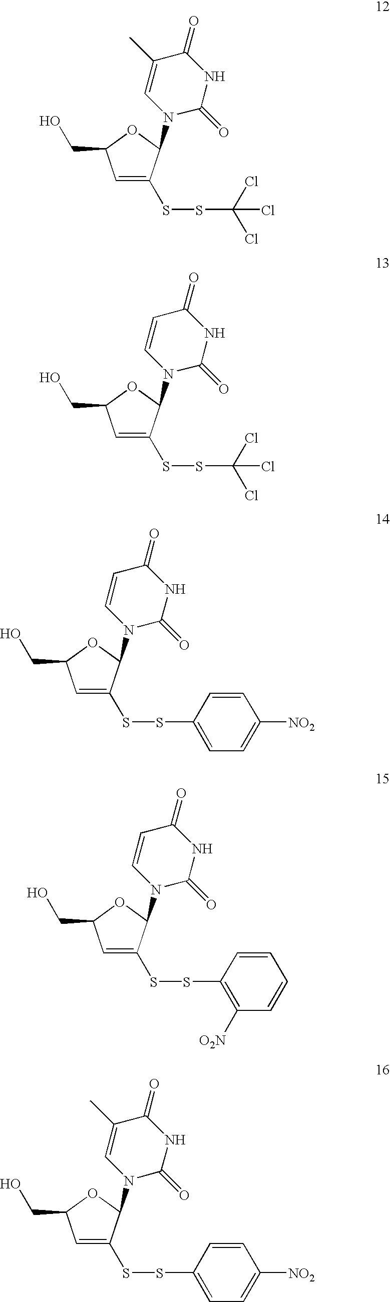

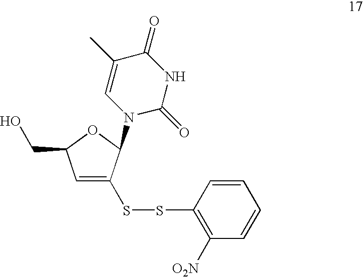

### Compounds of Formula (III)

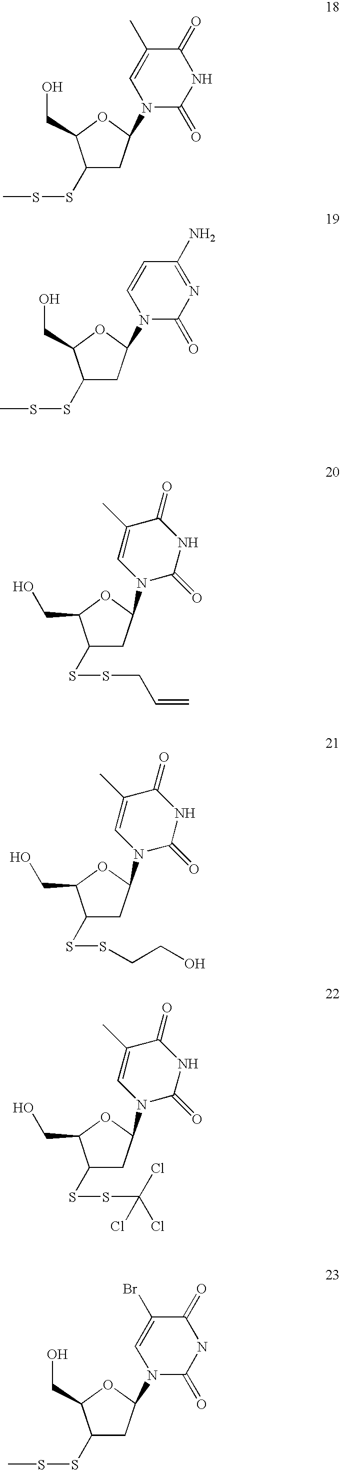

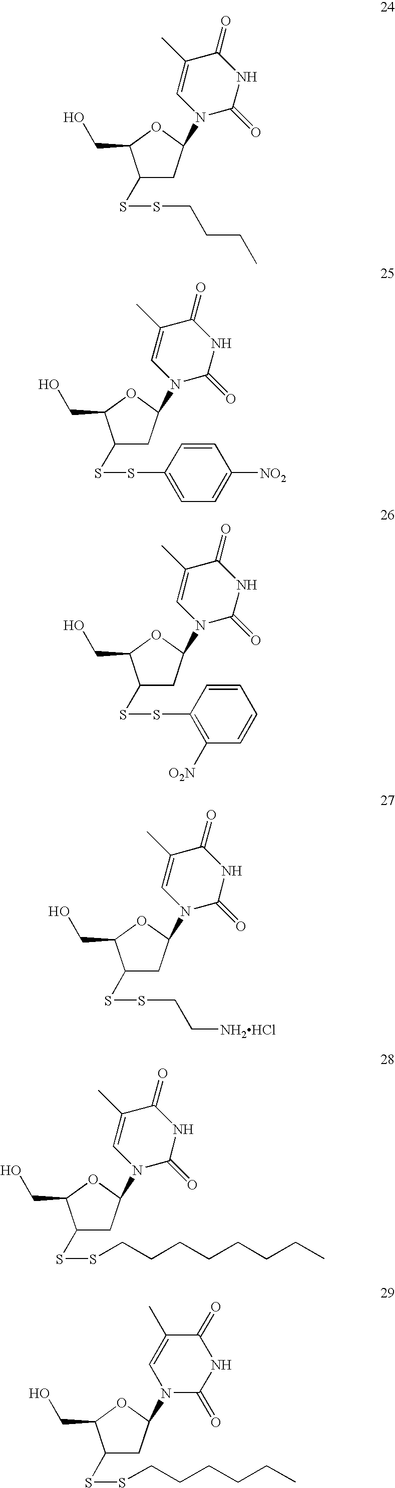

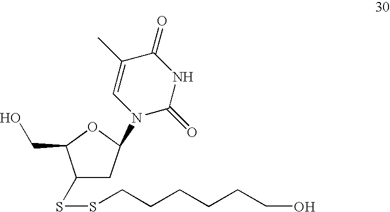

### Other Compounds of the Invention:

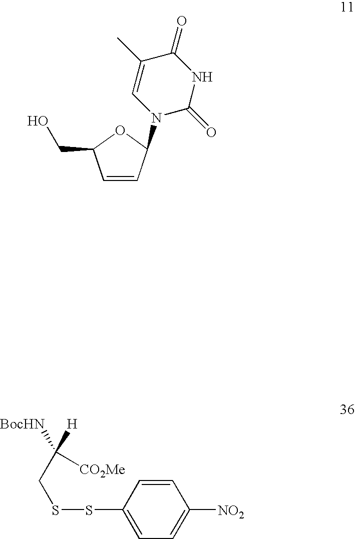

1. A nucleoside disulfide compound corresponding to formula (I) below: 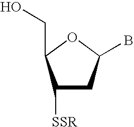
in which:
B represents thymine, and R is chosen from CH₂—CH═CH₂, C₄H₉, CH₂CH₂OH and CH₂CH₂NH₂.HCl.

2. A pharmaceutical composition comprising, as active principle, at least one compound chosen from:
the compounds corresponding to formula (II) below: 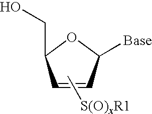
in which the S atom is bonded to the 2′ carbon or to the 3′ carbon of the nucleoside,
and the compounds corresponding to formula (III) below: 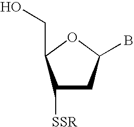
in which formulae
B represents a natural or modified purine or pyrimidine nucleotide base,
x is equal to 0, 1 or 2, and
R1 and R represent a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen,
and a pharmaceutically acceptable excipient.

3. The composition as claimed in claim 2, characterized in that the compound corresponds to formula (II) in which R1 is chosen from R3-Si(R4)(R5)(R6) in which R3 represents a hydrocarbon-based chain of two carbon atoms, which may be unsaturated and/or substituted, and R4, R5 and R6, which may be identical or different, each independently represent a hydrocarbon-based group.

4. The composition as claimed in claim 3, characterized in that R3 represents CH₂—CH₂ and R4, R5 and R6 are identical and represent CH₃.

5. The composition as claimed in claim 3 or 4, characterized in that B is chosen from natural or modified pyrimidine bases.

6. The composition as claimed in any one of claims 3 to 5, characterized in that the S atom is bonded to the 2′ carbon of the nucleoside.

7. The composition as claimed in any one of claims 3 to 6, characterized in that the compound corresponds to formula (II) in which x is equal to 0.

8. The composition as claimed in claim 7, characterized in that the compound is chosen from the following compounds:
2′,3′-Didehydro-2′,3′-dideoxy-2′-(2-trimethylsilyl)ethylthiouridine 1
2′,3′-Didehydro-2′,3′-dideoxy-2′-(3-methylbutyl)thiouridine 2
2′,3′-Didehydro-2′,3′-dideoxy-2′-(2-(trimethylsilyl)ethylthiothymidine 3
2′,3′-Didehydro-2′,3′-dideoxy-3′-(2-trimethylsilyl)ethylthiothymidine 4
2′,3′-Didehydro-2′,3′-dideoxy-2′-(2-trimethylsilyl)ethylthiocytidine 5

9. The composition as claimed in any one of claims 3 to 6, characterized in that the compound corresponds to formula (II) in which x is equal to 1.

10. The composition as claimed in claim 9, characterized in that the compound is chosen from the following compounds:
2′,3′-didehydro-2′,3′-dideoxy-2′-(2-trimethylsilyl)ethylthiothymidine sulfoxide 6
2′,3′-didehydro-2′,3′-dideoxy-2′-(2-trimethylsilyl)ethylthiouridine sulfoxide 8.

11. The composition as claimed in any one of claims 3 to 6, characterized in that the compound corresponds to formula (II) in which x is equal to 2.

12. The composition as claimed in claim 11, characterized in that the compound is 2′,3′-didehydro-2′,3′-dideoxy-2′-(2-trimethylsilyl)ethyl)thiothymidine sulfone 7.

13. The composition as claimed in claim 3, characterized in that x is equal to 0 and R1 represents SR2 in which R2 represents a carbon-based group or a hydrocarbon-based molecular residue that may be substituted and/or interrupted with atoms and/or with one or more groups comprising one or more atoms, said atoms being chosen from N, O, P, S, Si and X in which X represents a halogen.

14. The composition as claimed in claim 13, characterized in that R2 is chosen from ortho-nitrophenyl, para-nitrophenyl and trichloromethyl groups.

15. The composition as claimed in claim 13 or 14, characterized in that B is chosen from natural or modified pyrimidine bases.

16. The composition as claimed in any one claims 13 to 15, characterized in that the S atom is bonded to the 2′ carbon of 2′,3′-didehydro-2′,3′-dideoxyribose.

17. The composition as claimed in claim 16, characterized in that the compound is chosen from:
2′,3′-didehydro-2′,3′-dideoxythymidin-2′-yl trichloromethyl disulfide 12
2′,3′-didehydro-2′,3′-dideoxyuridin-2′-yl trichloromethyl disulfide 13
2′,3′-didehydro-2′,3′-dideoxythymidin-2′-yl 4-nitrophenyl disulfide 14
2′,3′-didehydro-2′,3′-dideoxythymidin-2′-yl 2-nitrophenyl disulfide 15
2′,3′-didehydro-2′,3′-dideoxyuridin-2′-yl 4-nitrophenyl disulfide 16
2′,3′-didehydro-2′,3′-dideoxyuridin-2′-yl 2-nitrophenyl disulfide 17.

18. The composition as claimed in claim 2, characterized in that the compound corresponds to formula (III) in which R is chosen from CH₂—CH═CH₂, C₄H₉, CH₂CH₂OH and CH₂CH₂NH₂.HCl, and ortho-nitrophenyl, para-nitrophenyl and trichloromethyl groups.

19. The composition as claimed in claim 18, characterized in that B represents a natural or modified pyrimidine base.

20. The composition as claimed in claim 19, characterized in that the compound is chosen from the following compounds:
3′-deoxythymidin-3′-yl allyl disulfide 20
3′-deoxythymidin-3′-yl 2-hydroxyethyl disulfide 21
3′-deoxythymidin-3′-yl trichloromethyl disulfide 22
3′-deoxythymidin-3′-yl butyl disulfide 24
3′-deoxythymidin-3′-yl 4-nitrophenyl disulfide 25
3′-deoxythymidin-3′-yl 2-nitrophenyl disulfide 26
3′-deoxythymidin-3′-yl 2-aminoethyl disulfide hydrochloride 27
3′-deoxythymidin-3′-yl hexyl disulfide 31
3′-deoxythymidin-3′-yl octyl disulfide 30
3′-deoxythymidin-3′-yl 6-hydroxyhexyl disulfide 32
2′,3′-dideoxycytidin-3′-yl methyl disulfide 19
5-bromo-2′,3′-dideoxyuridin-3′-yl methyl disulfide 23

21. The use of a compound chosen from:
the compounds corresponding to formula (II) below: 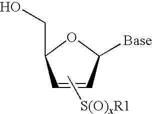
in which the S atom is bonded to the 2′ carbon or to the 3′ carbon of the nucleoside,
and the compounds corresponding to formula (III) below: 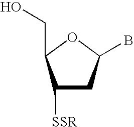
in which formulae
B represents a natural or modified purine or pyrimidine nucleotide base,
x is equal to 0, 1 or 2, and
R1 and R represent a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen,
as therapeutic active principle.

22. The use as claimed in claim 21, characterized in that the compound is defined in any one of claims 3 to 20.

23. The use of a compound chosen from:
the compounds corresponding to formula (II) below: 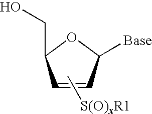
in which the S atom is bonded to the 2′ carbon or to the 3′ carbon of the nucleoside,
and the compounds corresponding to formula (III) below: 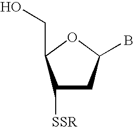
in which formulae
B represents a natural or modified purine or pyrimidine nucleotide base,
x is equal to 0, 1 or 2, and
R1 and R represent a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen,
for obtaining a medicament in an anticancer treatment.

24. The use as claimed in claim 23, characterized in that the compound is as defined in any one of claims 3 to 17.

25. The use as claimed in claim 23 or 24, characterized in that said compound has an anti-proliferative effect and/or a cytotoxic effect.

26. The use of a compound chosen from:
the compounds corresponding to formula (II) below: 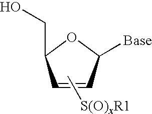
in which the S atom is bonded to the 2′ carbon or to the 3′ carbon of the nucleoside,
and the compounds corresponding to formula (III) below: 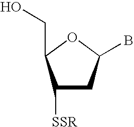
in which formulae
B represents a natural or modified purine or pyrimidine nucleotide base,
x is equal to 0, 1 or 2, and
R1 and R represent a carbon-based group or a hydrocarbon-based molecular residue that may be substituted and/or interrupted with one or more atoms and/or with one or more groups comprising one or more atoms, said atoms being chosen from N, O, P, S, Si and X in which X represents a halogen,
for obtaining a medicament in an antiviral treatment.

27. The use as claimed in claim 26, characterized in that the antiviral treatment is an anti-HIV1 or anti-HIV2 treatment.

28. The use as claimed in claim 26 or 27, characterized in that the compound is as defined in any one of claims 18 to 20.
